(19) United States
(12) Patent Application Publication
(10) Pub. No.: US 2026/0165704 A1
(43) Pub. Date: Jun. 18, 2026

(54) LINEAR SURGICAL STAPLER WITH MIS-CLAMP FEATURES

(71) Applicant: Cilag GmbH International, Zug (CH)

(72) Inventors: Andrew C. Deck, Cincinnati, OH (US); Brian D. Schings, Maineville, OH (US); Brett A. Rich, Crestview Hills, KY (US); David Norvell, Monroe, OH (US)

(21) Appl. No.: 18/980,062

(22) Filed: Dec. 13, 2024

(51) Int. Cl. A61B 17/068; A61B 17/072

(52) U.S. Cl. CPC A61B 17/0682; A61B 17/07207; A61B 2017/07264; A61B 2017/07285

(57) ABSTRACT

A surgical stapler includes a first elongate body having configured to present a first stapling surface, and a second elongate body configured to present a second stapling surface, the elongate bodies being configured to releasably couple together to enable the first and second stapling surfaces to cooperate to clamp and staple tissue with a plurality of staples. A clamp protrusion extends from the first elongate body, and a clamp lever is movably coupled with the second elongate body and includes an opening configured to releasably capture and draw the clamp protrusion toward the second elongate body as the clamp lever advances from an open position toward a closed position to thereby approximate the first and second stapling surfaces. A blocking protrusion is positioned to block the clamp protrusion from translating distally relative to the first elongate body when the clamp protrusion is misaligned with the opening of the clamp lever.

### BACKGROUND

In some surgical operations, such as a gastrointestinal anastomosis, it may be desirable to clamp down on one or more layers of tissue, cut through the clamped layers, and simultaneously drive staples through the layers to substantially seal the severed layers together near their severed ends. One such instrument that may be used in such operations is a linear surgical stapler, also referred to as a “linear cutter.” A linear surgical stapler generally includes a first half (referred to as a “cartridge half” or “reload half”) having a distal jaw configured to support a staple cartridge (or “reload”), and a second half (referred to as an “anvil half”) having a distal jaw that supports an anvil surface having staple forming features. The stapler further includes a moveable clamp lever configured to releasably clamp the stapler halves together. The stapler halves are configured to releasably couple together and pivot relative to one another to clamp tissue positioned between the two distal jaws when the clamp lever is closed. A firing assembly of the stapler is configured to be actuated to cut the clamped layers and simultaneously drive staples through the tissue on either side of the cut line. After the stapler is fired, the clamp lever may be opened, and the stapler halves separated to release the severed and stapled tissue.

While various kinds of surgical stapling instruments and associated components have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a perspective view of an illustrative linear surgical stapler, showing a cartridge half and an anvil half of the stapler coupled together with a clamp lever of the cartridge half in a fully closed position;

FIG. 2 depicts an exploded perspective view of the linear surgical stapler of FIG. 1, additionally showing a staple cartridge;

FIG. 3 depicts a perspective view of a distal end portion of the anvil half of the linear surgical stapler of FIG. 1;

FIG. 4 depicts a perspective view of a distal end portion of the staple cartridge of FIG. 2;

FIG. 5 depicts a cross-sectional perspective view of a proximal portion of the cartridge half of the linear surgical stapler of FIG. 1 with the clamp lever in an open position to reveal details of a firing assembly and a retaining assembly of the cartridge half;

FIG. 6 depicts an exploded perspective view of the retaining assembly of FIG. 5;

FIG. 7 depicts another exploded perspective view of the retaining assembly of FIG. 5;

FIG. 8 depicts a perspective view of the firing assembly of FIG. 5;

FIG. 9 depicts a perspective view of a staple cartridge assembly that includes the staple cartridge of FIG. 2;

FIG. 10 depicts an exploded perspective view of the staple cartridge assembly of FIG. 9;

FIG. 11 depicts a top plan view of the staple cartridge of FIG. 9;

FIG. 12 depicts a rear elevational view of the staple cartridge of FIG. 9;

FIG. 13 depicts an enlarged perspective view of a distal portion of the staple cartridge of FIG. 9, showing tissue gripping members formed on a deck of the staple cartridge;

FIG. 14 depicts an enlarged perspective view of a deck portion of the staple cartridge of FIG. 9, showing bridge elements of the tissue gripping members and showing staple drivers and staples of the staple cartridge in an actuated state;

FIG. 15 depicts a perspective view of a staple driver unit and a corresponding pair of staples of the staple cartridge of FIG. 9;

FIG. 16 depicts a side elevational view of the staple cartridge of FIG. 9;

FIG. 17 depicts a side elevational view of another illustrative staple cartridge configured for use with a linear surgical stapler;

FIG. 18 depicts a side elevational view of another illustrative staple cartridge configured for use with a linear surgical stapler;

FIG. 19A depicts a side elevational view of the linear surgical stapler of FIG. 1, showing the stapler halves separated from one another with the clamp lever in the open position;

FIG. 19B depicts a side elevational view of the linear surgical stapler of FIG. 1, showing proximal ends of the stapler halves coupled together while the clamp lever is in the open position to provide the stapler in a “hang-open” state;

FIG. 19C depicts a side elevational view of the linear surgical stapler of FIG. 1, showing distal portions of the stapler halves having been approximated so that a distal pin of the anvil half is received by clamp lever jaws of the cartridge half;

FIG. 19D depicts a side elevational view of the linear surgical stapler of FIG. 1, showing closure of the clamp lever to fully clamp the stapler halves together;

FIG. 19E depicts a side elevational view of the linear surgical stapler of FIG. 1, showing distal actuation of the firing assembly while the stapler halves are in the fully clamped state;

FIG. 20 depicts a perspective view of a proximal portion of another illustrative linear surgical stapler, showing a cartridge half and an anvil half of the stapler coupled together with a clamp lever of the cartridge half in a fully closed position with a latch of the clamp lever in a latched state;

FIG. 21 depicts a perspective view of a clamp lever latch of the linear surgical stapler of FIG. 20;

FIG. 22 depicts a side cross-sectional view of the proximal portion of the linear surgical stapler of FIG. 20, showing details of a firing assembly, a retaining assembly, and the clamp lever latch of the cartridge half, showing the clamp lever in the fully closed position with the latch in an unlatched state while the firing assembly is displaced distally from a proximal home position;

FIG. 23 depicts a side cross-sectional view of the proximal portion of the linear surgical stapler of FIG. 20, showing the latch in the latched state while the firing assembly is in the proximal home position;

FIG. 24 depicts a side elevational view of the proximal portion of the linear surgical stapler of FIG. 20, showing a protrusion located at a proximal end of a shroud of the anvil half and contacting a movable retainer of the cartridge half to resist decoupling of the proximal ends of the stapler halves when the stapler is in the hang-open state;

FIG. 25A depicts a side elevational view of another illustrative linear surgical stapler, showing a clamp lever of the stapler in a fully open position;

FIG. 25B depicts a side elevational view of the linear surgical stapler of FIG. 25A, showing the clamp lever in a partially closed, non-clamped position;

FIG. 25C depicts a side elevational view of the linear surgical stapler of FIG. 25A, showing the clamp lever in a fully closed, non-closed position;

FIG. 26 depicts a perspective view of another illustrative linear surgical stapler, showing a clamp lever of the stapler in a fully open position;

FIG. 27 depicts an enlarged side elevational view of a cartridge channel of the linear surgical stapler of FIG. 26, with other components of the linear surgical stapler omitted from view, showing a blocking protrusion defined by a distal sidewall portion of the cartridge channel; and

FIG. 28 depicts an enlarged side elevational view of the linear surgical stapler of FIG. 26, showing the clamp lever in a partially-closed, non-clamped position in which a clamp protrusion of the anvil half is misaligned with jaw slots of the clamp lever such that the clamp lever is inhibited from assuming a fully-closed position.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for illustrative description purposes only and are not intended to be limiting or absolute. In that regard, it will be understood that surgical instruments such as those disclosed herein may be used in a variety of orientations and positions not limited to those shown and described herein.

Furthermore, the terms “about” and “approximately” as used herein in connection with any numerical values or ranges indicate a suitable dimensional tolerance that allows the referenced feature(s) to function for its intended purpose as described herein.

### I. Illustrative Linear Surgical Staplers

### A. Overview of Linear Surgical Stapler

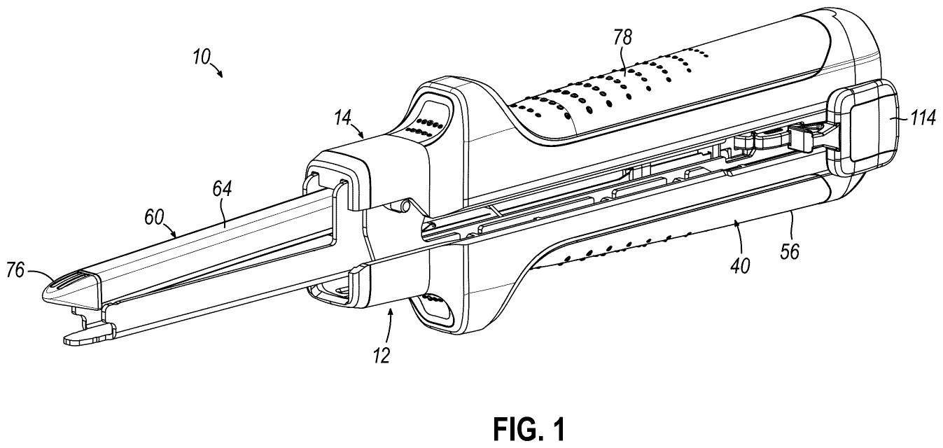

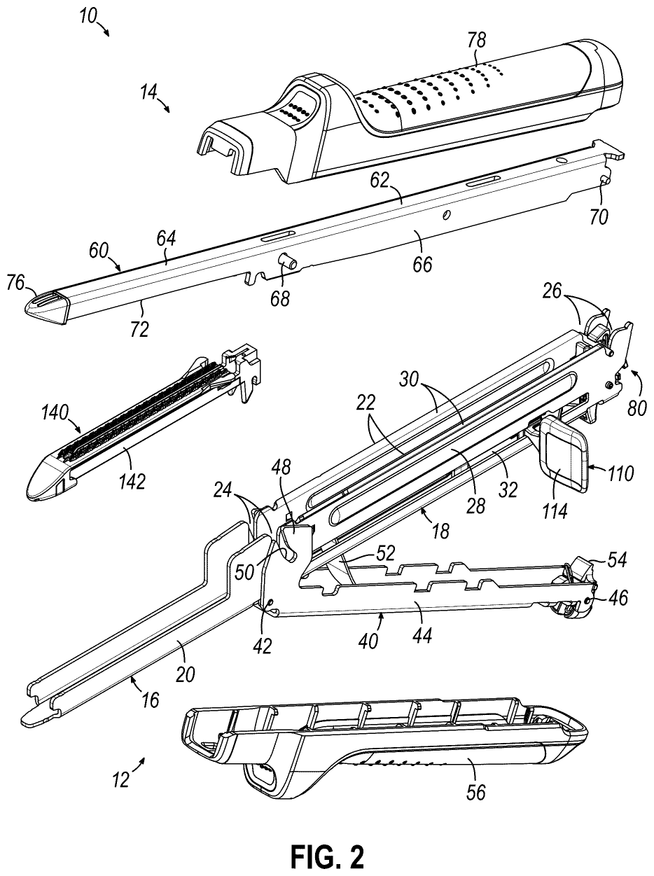

FIGS. 1-2 show an illustrative linear surgical stapler (10) (also referred to as a “linear cutter”) suitable for use in a variety of cutting and stapling procedures, such as a gastrointestinal anastomosis procedure. Linear surgical stapler (10) includes a cartridge half (12) (also referred to as a “reload half”) and an anvil half (14) configured to releasably couple together to clamp tissue therebetween for simultaneous cutting and stapling of the clamped tissue.

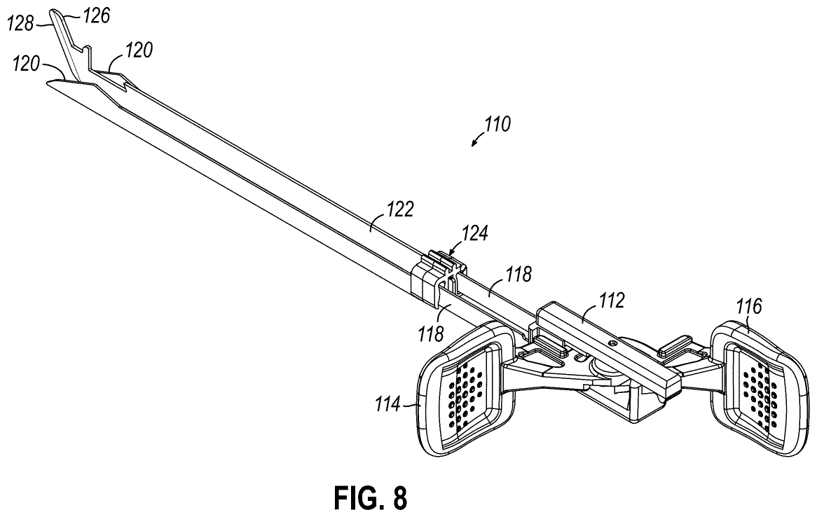

Cartridge half (12) includes a first elongate body in the form of an elongate cartridge channel (16) having a proximal frame portion (18) and a distal jaw portion (20). Proximal frame portion (18) slidably retains a firing assembly (110) and includes a laterally opposed pair of upright sidewalls (22). Each sidewall (22) includes a distal pin-receiving opening in the form of a vertical slot (24) arranged at a distal end thereof, and a proximal pin-receiving opening in the form of a tapered notch (26) arranged at a proximal end thereof. An outwardly projecting stiffening rib (28) extends longitudinally between the distal slot (24) and proximal notch (26) of each sidewall (22) and is configured to provide the sidewall (22) with enhanced stiffness. An outwardly flared upper segment (30) defines an upper edge of a proximal portion of each sidewall (22) and is configured to facilitate receipt of anvil half (14) by cartridge half (12). Each sidewall (22) further includes an elongate firing slot (32) extending longitudinally between proximal notch (26) and distal slot (24) along a lower side of sidewall (22). Elongate firing slots (32) are configured to guide firing assembly (110) between proximal and distal positions. Firing assembly (110) is described in greater detail below in connection with FIG. 8.

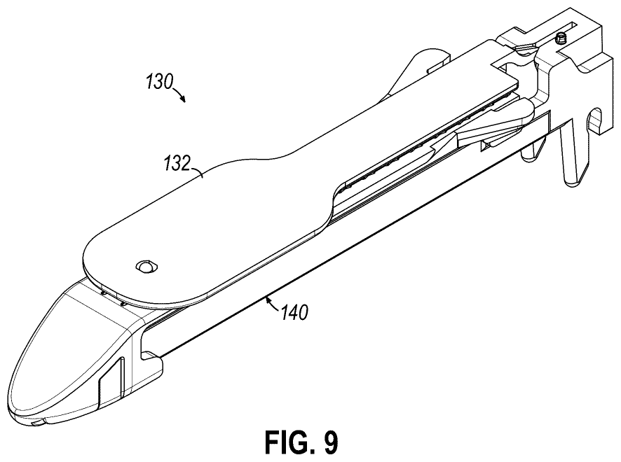

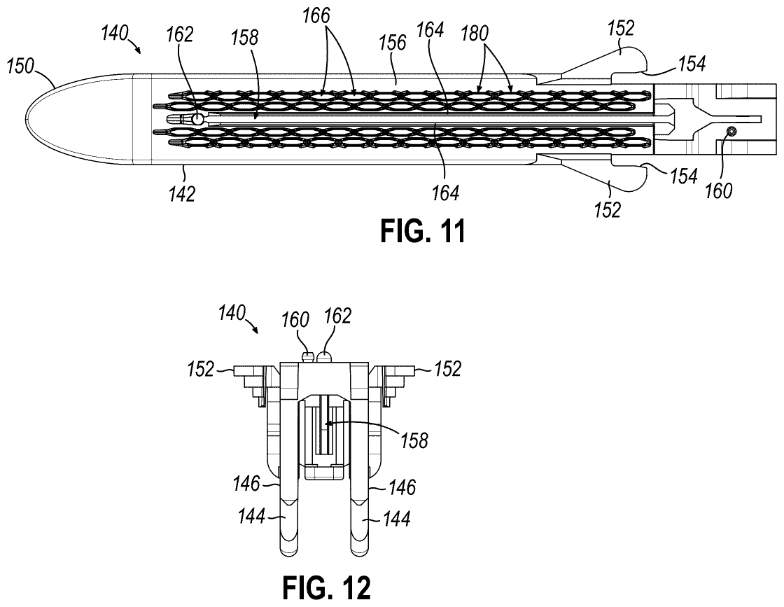

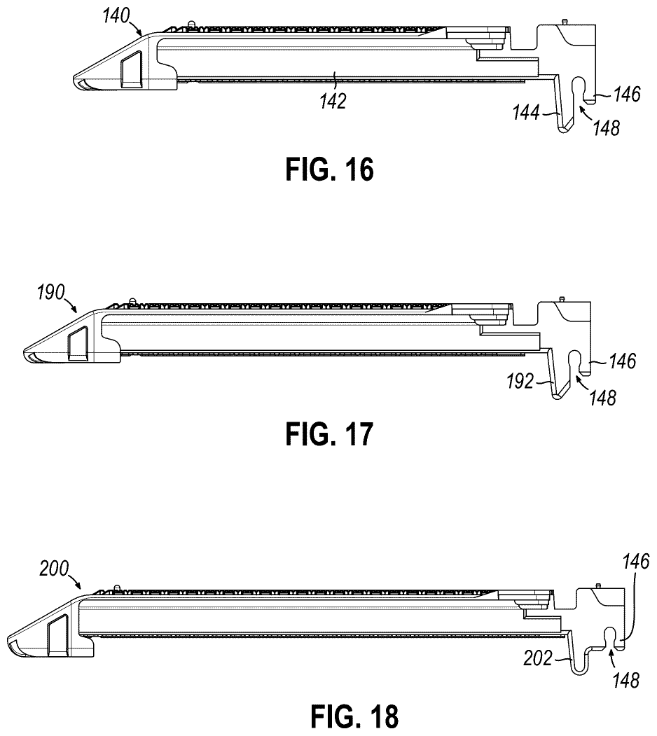

Distal jaw portion (20) of cartridge channel (16) is configured to releasably receive a stapling assembly in the form of a staple cartridge (140) (or “reload”). As shown in FIGS. 4 and 9-16, staple cartridge (140) includes a cartridge body (142) having an upper side that defines a stapling surface in the form of a deck (156) having a plurality of staple openings (166) that house a plurality of staples (168) and corresponding staple drivers (172) (see FIG. 16). Illustrative features of staple cartridge (140) are described in greater detail below in connection with FIGS. 9-16.

Cartridge half (12) further includes a clamp member in the form of a clamp lever (40) (also referred to as a “latch lever”) pivotably coupled to cartridge channel (16) with a clamp lever pivot pin (42), which is arranged in approximate alignment with distal slots (24) of cartridge channel sidewalls (22). Clamp lever (40) includes an elongate lever arm (44) having a free proximal end (46) and a distal end that is pivotably coupled to a lower portion of cartridge channel (16) with pivot pin (42). A pair of opposed jaws (48) extend distally from the distal end of lever arm (44) alongside cartridge channel sidewalls (22). Each jaw (48) includes a pin-receiving opening in the form of an arcuate slot (50) having a closed proximal end and an open distal end configured to receive a clamp pin (68) (also referred to as a latch pin) of anvil half (14), as described below.

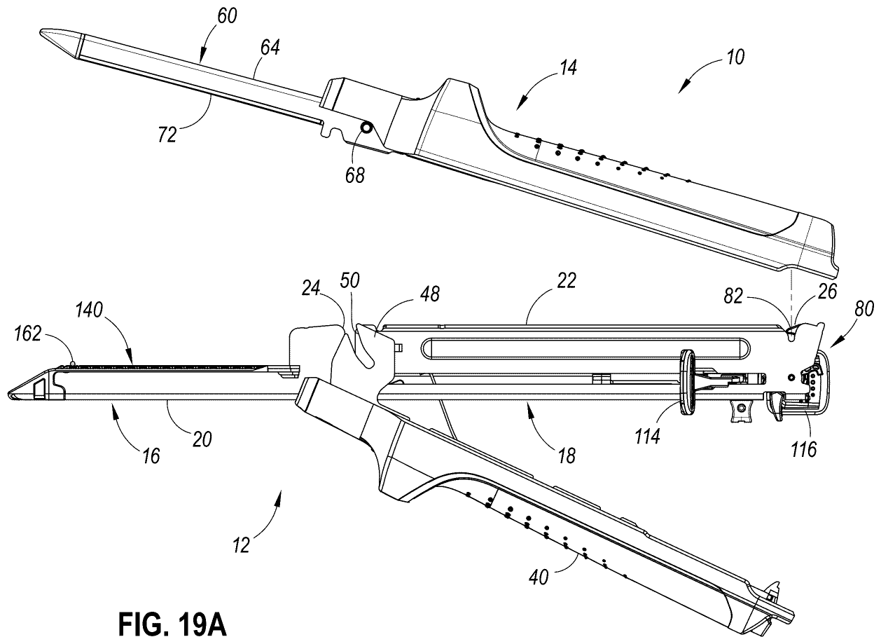

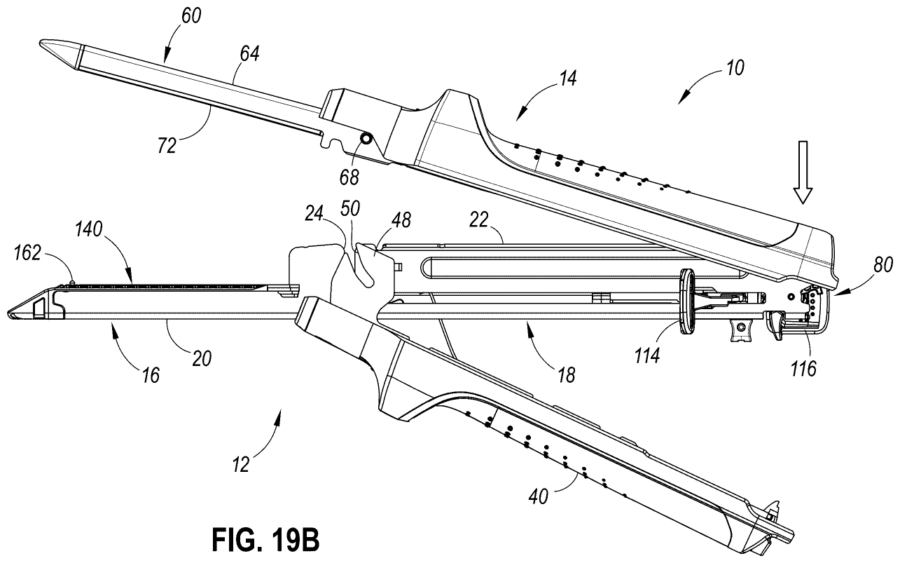

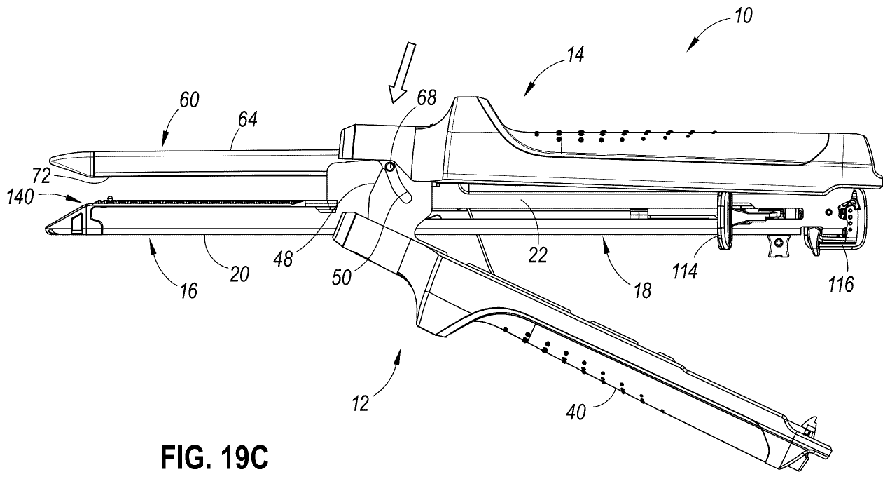

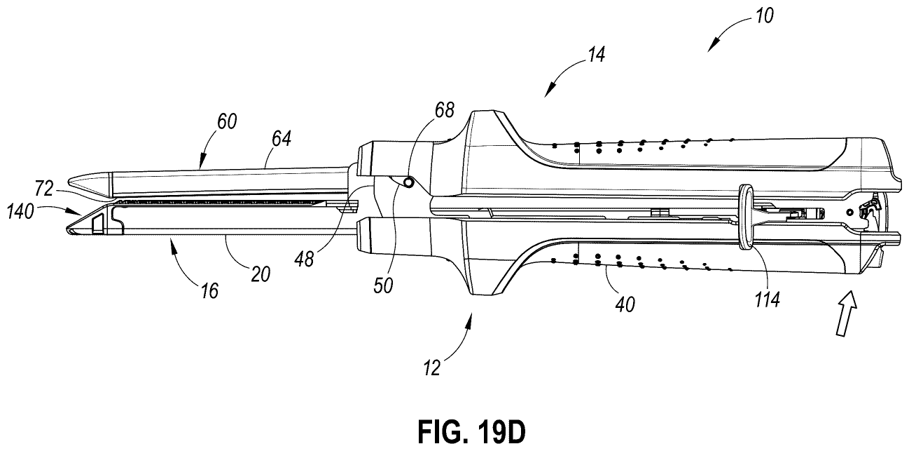

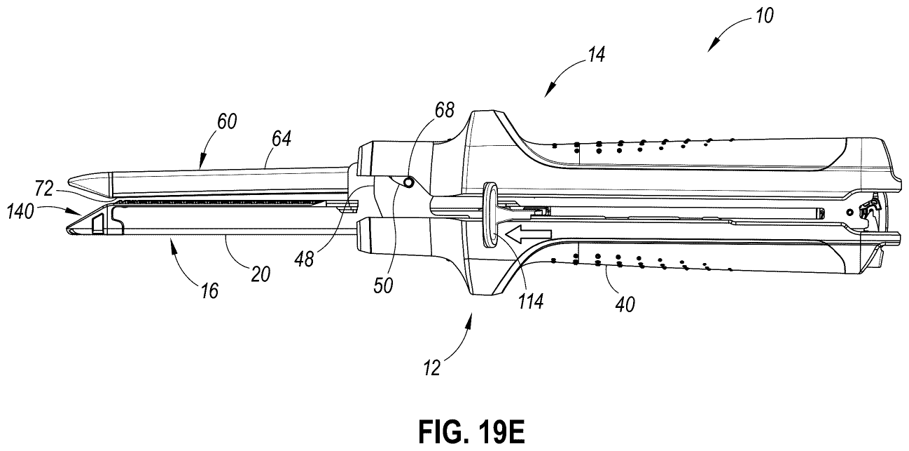

Clamp lever (40) is operable to pivot relative to cartridge channel (16) between an open position in which proximal end (46) of lever arm (44) is spaced from cartridge channel frame portion (18) as shown in FIGS. 19A-19C described below, and a closed position in which proximal end (46) confronts cartridge channel frame portion (18) as shown in FIG. 9D described below. Actuation of clamp lever (40) from the open position to the closed position operates to capture the opposed lateral ends of clamp pin (68) within clamp lever jaw slots (50), and thereby clamp anvil half (14) against cartridge half (12), as shown and described below in connection with FIGS. 19C-19D. In that regard, the curvature of each jaw slot (50) defines respective upper and lower camming surfaces configured to engage and draw the respective lateral end of clamp pin (68) toward cartridge channel (16) as clamp lever (40) is pivotably closed. A resilient member shown in the form of a leaf spring (52) biases lever arm (44) toward the open position. Accordingly, leaf spring (52) promotes disengagement of clamp lever jaws (48) from anvil half clamp pin (68) upon initial advancement of clamp lever (40) from the closed position toward the open position.

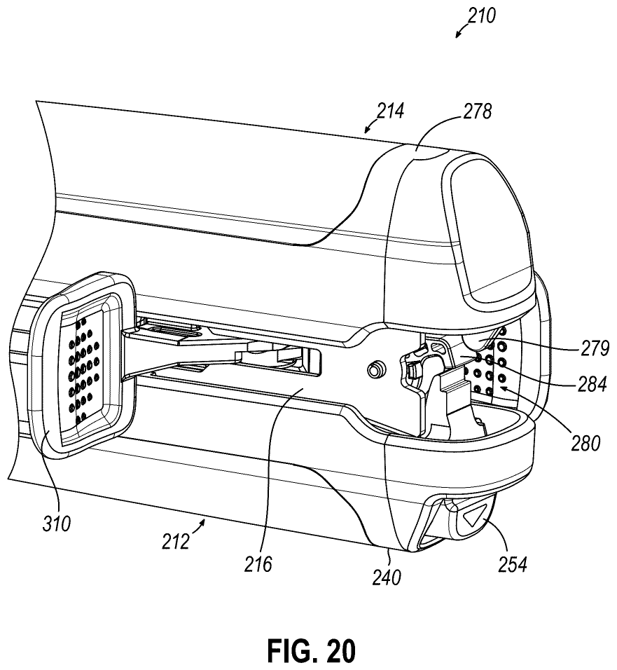

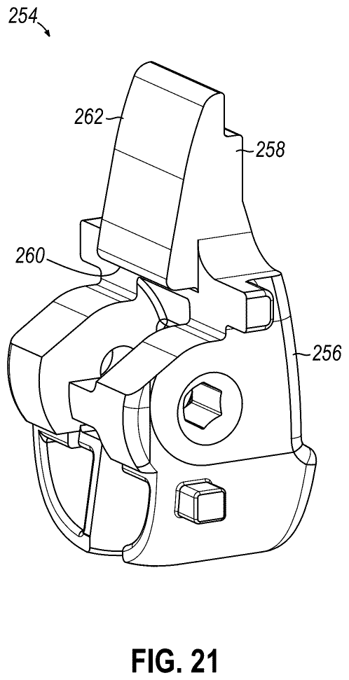

As best shown in FIG. 2, clamp lever (40) further includes a latch (54) arranged at proximal end (46) of lever arm (44). Clamp lever latch (54) is configured to resiliently and releasably engage a proximal end of cartridge channel frame portion (18) and thereby releasably retain clamp lever (40) in the closed position, for instance while stapler (10) is being fired. Clamp lever latch (54) may be further configured in accordance with the teachings of U.S. Pat. No. 11,278,285, entitled “Clamping Assembly for Linear Surgical Stapler,” issued Mar. 22, 2022, the disclosure of which is incorporated by reference herein. An illustrative variation of clamp lever latch (54) is described in greater detail below in connection with FIGS. 20-23.

Anvil half (14) of linear surgical stapler (10) includes a second elongate body in the form of an elongate anvil channel (60) having a proximal frame portion (62) and a distal jaw portion (64). Proximal frame portion (62) includes a laterally opposed pair of upright sidewalls (66) that are configured to be received between cartridge channel sidewalls (22) when anvil half (14) is coupled with cartridge half (12). A fixed distal clamp protrusion in the form of clamp pin (68) extends laterally through the distal ends of anvil channel sidewalls (66), and a fixed proximal latch protrusion in the form of proximal pin (70) extends laterally through the proximal ends of anvil channel sidewalls (66). Anvil half pins (68, 70) are configured to facilitate coupling of anvil half (14) with cartridge half (12), as described below.

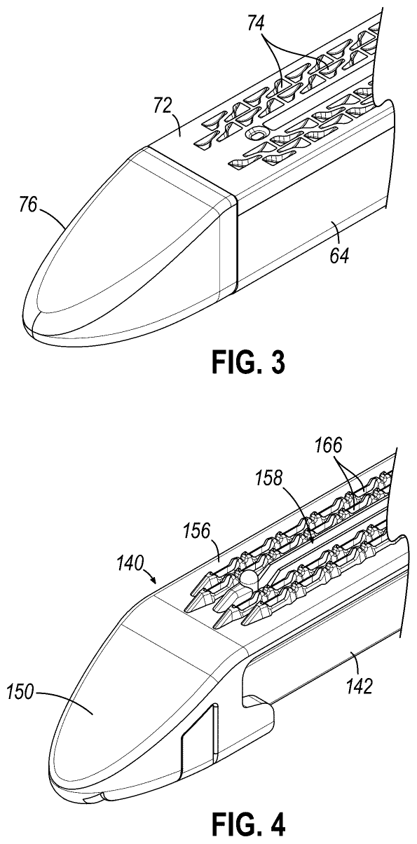

As shown in FIGS. 2 and 3, distal jaw portion (64) of anvil half (14) supports an anvil plate (72) that defines a stapling surface in the form of an anvil surface having a plurality of staple forming pockets (74) configured to deform legs of staples ejected by staple cartridge (140) when stapler (10) is fired. Staple forming pockets (74) of the present example may be formed via a coining process and are configured to form each staple of staple cartridge (140) with a three-dimensional shape in which the legs of each formed staple are laterally offset from one another so as to provide the formed staple with a non-planar shape, for example as disclosed in U.S. Pat. No. 11,229,433, entitled “Linear Surgical Stapler,” issued Jan. 25, 2022, the disclosure of which is incorporated by reference herein. Anvil channel (60), anvil plate (72), and staple forming pockets (74) may be formed in one or more of the manners disclosed in U.S. Pat. Nos. 11,229,433; 11,045,193, entitled “Anvil Assembly for Linear Surgical Stapler,” issued Jun. 29, 2021; and/or U.S. Pub. No. 2022/0142641, entitled “System and Method for Forming Pockets in Anvil of Surgical Stapler,” published May 12, 2022, the disclosures of which are incorporated by reference herein. For instance, distal jaw portion (64) of anvil half (14) may be pre-formed with a curvature along its length that accommodates deflection of distal jaw portion (64) and anvil plate (72) when stapler halves (12, 14) are clamped together by clamp lever (40). Distal jaw portion (64) of anvil half (14) additionally supports a tapered distal tip member (76). In some versions, distal tip member (76) may be selectively extendable relative to distal jaw portion (64) in accordance with the teachings of U.S. Pat. No. 11,033,266, entitled “Decoupling Mechanism for Linear Surgical Stapler, issued Jun. 15, 2021, the disclosure of which is incorporated by reference herein.

As shown in FIG. 2, linear surgical stapler (10) further includes a pair of shrouds (56, 78) that cover select portions of stapler (10) and promote effective grip and manipulation of stapler (10) by an operator during use. In the present example, a clamp lever shroud (56) is affixed to and covers an outwardly facing side of clamp lever (40) such that clamp lever shroud (56) is configured to pivot with clamp lever (40) relative to cartridge channel (16). Additionally, an anvil shroud (78) is affixed to and covers an outwardly facing side of anvil channel (60). In some versions, anvil shroud (78) may be coupled with anvil channel (60) via interaction between pins (68, 70) and one or more tabs, ribs, or other structures that are disposed within an interior of anvil shroud (78) and include an opening, slot, keyhole, or other feature configured to receive a respective one of pins (68, 70). By way of example only, shrouds (56, 78) may be affixed using one or more of the teachings of U.S. Pat. No. 11,278,285, incorporated by reference above. In other versions, shrouds (56, 78) may be coupled with clamp lever (40) and anvil channel (60) in a variety of other suitable manners readily apparent to those of ordinary skill in the art in view of the teachings herein.

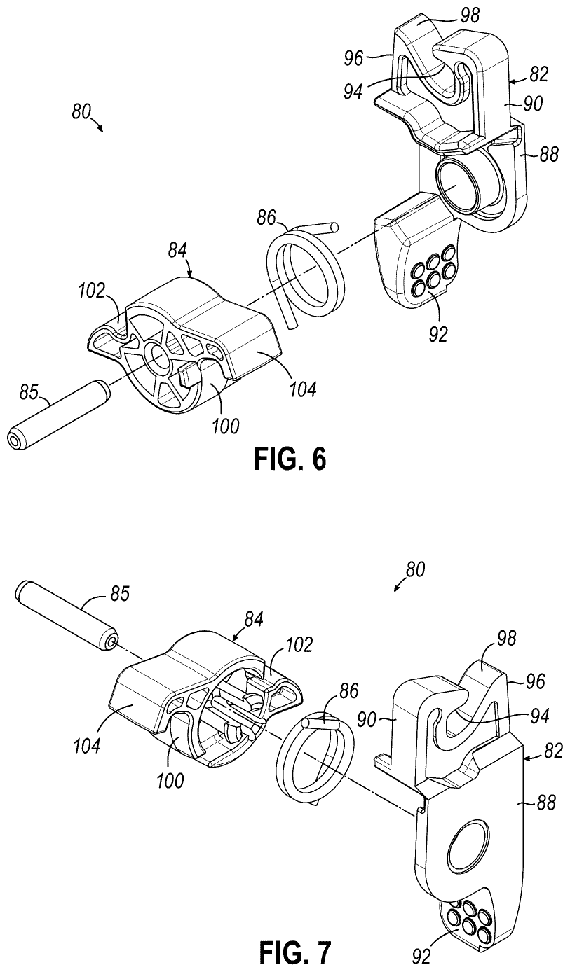

As shown best in FIGS. 2 and 5-7, a proximal end of cartridge half (12) includes a retaining assembly (80) configured to releasably retain portions of anvil half (14) and firing assembly (110). Retaining assembly (80) of the present example includes a first movable retainer in the form of an anvil latch (82) and a second movable retainer in the form of a detent (84). Anvil latch (82) and detent (84) are rotatably coupled with a proximal end of cartridge channel (16) via a laterally extending pin (85) arranged proximally of firing slots (32), and retainers (82, 84) are resiliently biased in opposite rotational directions by a resilient member in the form of a torsion spring (86) positioned between retainers (82, 84).

Anvil latch (82) includes a central body (88), a latch finger (90) extending upwardly from central body (88), and a release button (92) extending downwardly from central body (88) though a base wall of proximal frame portion (18) of cartridge channel (16). An upper end of latch finger (90) tapers distally and is configured to releasably capture proximal anvil pin (70) of anvil half (14) with an angled latching surface (94) that overlies proximal anvil pin (70) once captured. Anvil latch (82) further includes a pin ejection feature in the form of an angled protrusion (96) extending distally from a base portion of latch finger (90) and which defines an ejection cam ramp (98) that faces proximally toward latch finger (90).

Detent (84) of proximal retaining assembly (80) includes a generally cylindrical central body (100), a distal finger (102) extending distally from central body (100), and a proximal hook (104) extending proximally from central body (100). Distal finger (102) is configured to releasably engage a proximal end of firing assembly (110) and thereby retain firing assembly (110) in a proximal home position. Proximal hook (104) is configured to overlie and capture an upper tip of clamp lever latch (54) when clamp lever (40) is fully closed and firing assembly (110) is translated distally from its proximal home position, thereby preventing clamp lever (40) from opening during a firing stroke, for example as described in greater detail in U.S. Pat. No. 11,278,285, incorporated by reference above.

In use, with stapler halves (12, 14) coupled together at their proximal ends such that proximal anvil pin (70) is retained by anvil latch (82), and with clamp lever (40) in the open position, distal actuation of lower release button (92) causes anvil latch (82) to rotate about pin (85) such that ejection cam ramp (98) advances proximally to drive proximal anvil pin (70) upwardly out of proximal tapered notches (26) of cartridge channel (16). Cartridge half (12) of the present version further includes a stationary finger grip protrusion (106) that extends downwardly from a base wall of proximal frame portion (18) of cartridge channel (16) at a location distal to lower release button (92), and is configured to facilitate actuation of release button (92). In particular, a user may apply his or her thumb to a proximal side of release button (92) and one or more fingers to a distal side of finger grip protrusion (106), and then squeeze release button (92) distally toward stationary finger grip protrusion (106) to rotate latch finger (90) out of engagement with proximal anvil pin (70) and eject pin (70) upwardly from cartridge channel (16) with ejection cam ramp (98).

Retaining assembly (80) and related components of cartridge half (12) may be further configured and operable in accordance with one or more teachings of U.S. Pat. No. 10,898,187, entitled “Firing System for Linear Surgical Stapler,” issued Jan. 26, 2021, the disclosure of which is incorporated by reference herein; and/or U.S. Pat. No. 11,033,266, incorporated by reference above.

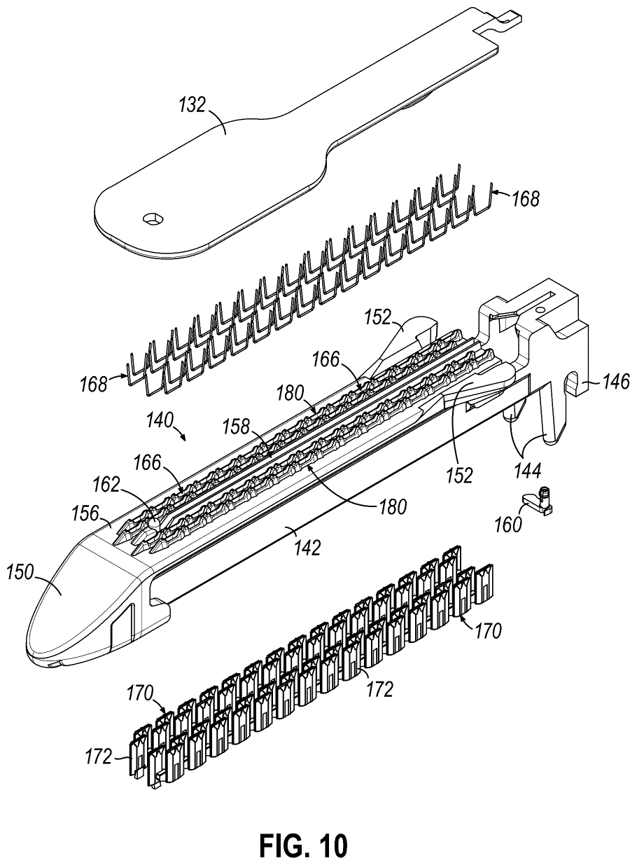

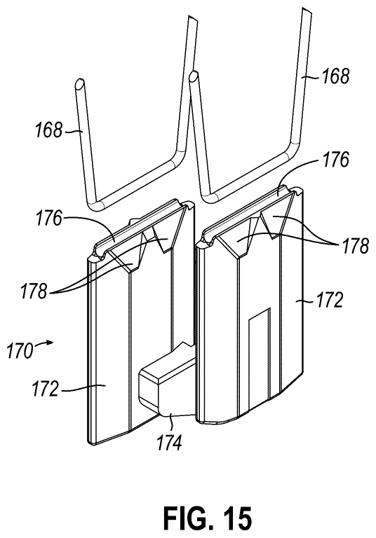

As shown in FIG. 8, firing assembly (110) of cartridge half (12) includes a slide block (112), a pair of actuators (114, 116) (or “firing knobs”) pivotably coupled to slide block (112), and a set of elongate beams (118, 122) extending distally from slide block (112). A pair of side beams (118) are coupled at their proximal ends to a distal end of slide block (112) and terminate distally in a pair of cam ramps (120). Cam ramps (120) are configured to engage the undersides of staple drivers (172) (see FIGS. 10 and 15) housed within staple cartridge (140) and actuate staple drivers (172) upwardly to thereby drive (or “fire”) staples from cartridge (130) into tissue clamped between staple cartridge (140) and anvil plate (72). A center beam (122) is coupled with side beams (118) via a bridge (124) (or “knife block”) spaced distally from slide block (112). Center beam (122) terminates distally in a distally angled knife (126) having a distal cutting edge (128) configured to cut tissue clamped between the distal portions of stapler halves (12, 14).

Each actuator (114, 116) of firing assembly (110) is configured and rotatable relative to slide block (112) between a deployed position and a retracted position such that only one actuator (114, 116) may be deployed at a time, for example as disclosed in U.S. Pat. No. 10,898,187, incorporated by reference above. In the deployed position, an actuator (114, 116) may be driven distally by an operator to actuate firing assembly (110) distally through stapler (10) and thereby simultaneously cut and staple tissue clamped between stapler halves (12, 14).

### B. Illustrative Staple Cartridges

FIGS. 9-16 show an illustrative staple cartridge assembly (130) configured for use with linear surgical stapler (10). Staple cartridge assembly (130) includes staple cartridge (140) and a retainer (132) configured to releasably couple with staple cartridge (140) to retain staples (168) in staple cartridge (140) before use.

Staple cartridge (140) includes a cartridge body (142) that extends linearly along a longitudinal axis between a proximal end having a cartridge channel coupling feature in the form of a plurality of downwardly extending coupling legs (144, 146) (also known as “fangs”), and a distal end having a tapered nose (150). Coupling legs (144, 146) are configured to releasably capture clamp lever pivot pin (42) and extend downwardly through corresponding openings formed in a floor of cartridge channel (16) when staple cartridge (140) is seated within distal jaw portion (20) of cartridge channel (16). A pair of wing tabs (152) disposed on the lateral sides of cartridge body (142) near the proximal end are configured to facilitate insertion and removal of staple cartridge (140) relative to distal jaw portion (20). As shown in FIG. 20, an interior side of each wing tab (152) includes a chamfer (154) at its proximal end that serves as a relief feature to provide clearance for the sidewalls of distal jaw portion (20) to facilitate installation and removal of staple cartridge (140) without interference.

An upper side of cartridge body (142) defines a deck (156). An elongate knife slot (158) extends longitudinally through deck (156) along the longitudinal axis of staple cartridge (140) and is configured to slidably receive knife (126) of firing assembly (110) therethrough in response to distal actuation thereof, described above. A firing lockout bypass feature in the form of a swing tab (160) is rotatably coupled to cartridge body (142) at a proximal end of knife slot (158). Swing tab (160) is configured to rotate between a deployed position in which swing tab (160) extends perpendicularly across the proximal end of knife slot (158), and a retracted position in which swing tab (160) extends parallel to knife slot (158). Swing tab (160) in the deployed position is configured to urge firing assembly (110) from a lockout state toward a firing state in which firing beams (118, 122) may translate distally through staple cartridge (140) to enact stapling and cutting of tissue clamped by stapler (10).

A rigid tissue gap post (162) is secured at a distal end of knife slot (158) and protrudes upwardly away from cartridge deck (156). A rounded upper end of tissue gap post (162) is configured to contact a distal end of anvil plate (72) and thereby define a tissue gap of predetermined size between cartridge deck (156) and anvil plate (72) when stapler halves (12, 14) are clamped together in the manner described above.

A pair of elongate ribs (164) extend along opposing sides of knife slot (158) and project away from deck (156) to define raised surfaces. Elongate ribs (164) terminate at proximal and distal ends of knife slot (158) and are configured to promote enhanced gripping of tissue along knife slot (158), thus stabilizing the tissue during cutting by knife (126).

Cartridge body (142) of staple cartridge (140) further includes a plurality of staple openings (166) that extend transversely through cartridge body (142) and open to deck (156). In the present example, staple openings (166) are arranged in first and second parallel rows along each side of knife slot (158), such that the staple openings (166) of each row are longitudinally staggered relative to staple openings (166) of the adjacent row. It will be understood that various suitable arrangements and quantities of staple openings (166) may be provided in other versions of staple cartridge (140). Each staple opening (166) is configured to house a respective staple (168) and staple driver (172). As described above, cam ramps (120) of firing assembly (110) are configured to engage the undersides of staple drivers (172) and actuate staple drivers (172) upwardly within staple openings (166) to drive (or “fire”) staples (170) from staple openings (166), into tissue, and against anvil plate (72).

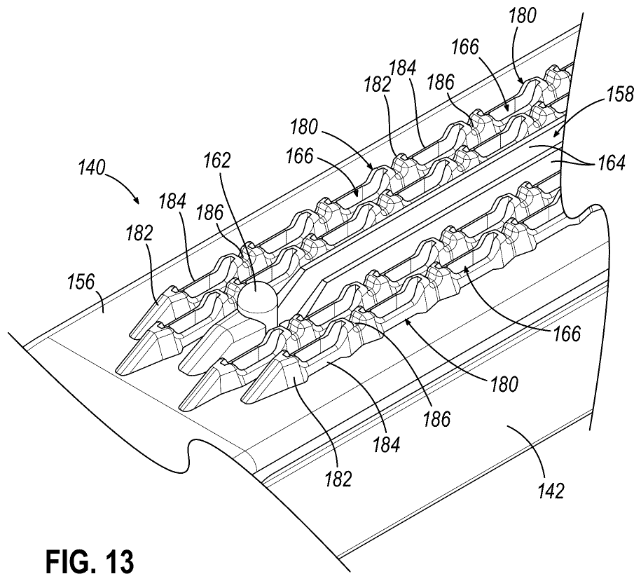

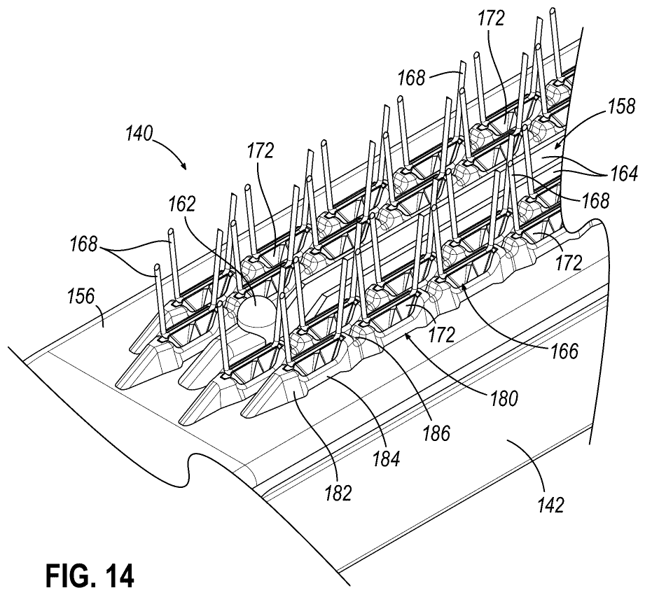

As shown best in FIGS. 13-14, staple cartridge (140) further includes a plurality of tissue gripping members (180) arranged on and projecting upwardly from cartridge deck (156). Tissue gripping members (180) are distributed along a length of deck (156) and are laterally offset from knife slot (158) and elongate ribs (164) to align with and open to a respective one or more staple openings (166). Tissue gripping members (180) are configured to grip and thereby stabilize tissue when deck (156) and anvil plate (72) are clamped together; and, further, optimize tissue compression at the staple locations to facilitate effective stapling and cutting of the tissue. Tissue gripping members (180) of the present version are integrally connected with cartridge body (142).

Each tissue gripping member (180) includes a first end feature (182) that wraps partially around a first end portion of a staple opening (166), and an opposed second end feature (182) that wraps partially around an opposed second end portion of the same staple opening (166). A pair of bridge elements (184) extend between the first and second end features (182) along opposed lateral sides of the staple opening (166) and are recessed relative to the first and second end features (182). Accordingly, each bridge element (184) has a maximum height relative to deck (156) that is less than a maximum height of each of the first and second end features (182). Each pair of bridge elements (184) is configured to cooperate with the respective end features (182) to compress and grip tissue around the respective staple opening (166), and to guide a respective staple driver (172) toward anvil plate (72) without contacting the legs of the respective staple (168) during staple leg formation. Such compression of tissue by end features (182) and bridge elements (184) assists in gripping and stabilizing the tissue, while also minimizing the compression of tissue by staple drivers (172) themselves, which in turn minimizes the user input force required to fire stapler (10). Additionally, at least one end feature (182) associated with each staple opening (166) is integrally connected with an adjacent end feature (182) associated with a longitudinally adjacent staple opening (166), with a recessed region (186) located between each integrally connected pair of end features (182).

As shown in FIG. 15, each pair of staple drivers (172) of staple cartridge (140) is integrally formed as a staple driver unit (170) having a bridge feature (174) that interconnects staple drivers (172) at their lower ends. Staple drivers (172) of driver unit (170) are arranged in a staggered formation in which staple drivers (172) are laterally and longitudinally offset from one another, relative to the longitudinal axis of staple cartridge (140), such that staple drivers (172) are configured to align with respective staple openings (166) of cartridge body (142). Each staple driver (172) includes a groove (176) at its upper end configured to receive and support the crown of a respective staple (168). Additionally, each staple driver (172) includes at its upper end staple leg receiving features in the form of pockets (178) that are defined by respective chamfered surfaces and are configured to receive staple leg tips of a respective staple (168) during formation of staple (168) by anvil plate (72) when stapler (10) is fired. Such features are described in greater detail in U.S. Pat. No. 11,229,433, incorporated by reference above.

As described above, coupling legs (144, 146) formed at the proximal end of staple cartridge (140) facilitate coupling of staple cartridge (140) with distal jaw portion (20) of cartridge channel (16). More specifically, each lateral side of staple cartridge (140) includes a longer distal coupling leg (144) and a shorter proximal coupling leg (146), separated by a gap (148). Gap (148) is suitably positioned and shaped, for example with an oval-like shape, to provide proximal coupling leg (146) with a smaller minimum thickness in a longitudinal direction than distal coupling leg (144), thus rendering proximal coupling leg (146) suitably flexible relative to cartridge body (142). Accordingly, during installation of staple cartridge (140) into distal jaw portion (20), proximal coupling leg (146) is configured to deflect relative to distal coupling leg (144) to enable clamp lever pivot pin (42) to be received into gap (148) with a snap-fit engagement and with a suitable degree of installation force by the user.

Distal coupling leg (144) of staple cartridge (140) may also serve as a poka-yoke feature that is sized and shaped to render staple cartridge (140) usable with only a predetermined type of linear surgical stapler, such as a stapler configured to apply to tissue a staple pattern and cut line of a predetermined length (e.g., 60 mm). FIG. 17 shows another illustrative staple cartridge (190) that is generally similar to staple cartridge (140), except that staple cartridge (190) is longer and has a uniquely shaped distal coupling leg (192) that renders staple cartridge (190) usable with only another predetermined type of linear surgical stapler, such as a stapler configured to apply a longer (e.g., 80 mm) staple pattern and cut line to tissue. FIG. 18 shows yet another illustrative staple cartridge (200) that is generally similar to staple cartridges (140, 190), except that staple cartridge (200) is even longer and has a uniquely shaped distal coupling leg (202) that renders staple cartridge (200) usable with only another predetermined type of linear surgical stapler, such as a stapler configured to apply an even longer (e.g., 100 mm) staple pattern and cut line to tissue.

Staple cartridges (140, 190, 200) may be further configured in accordance with the teachings of U.S. patent application Ser. No. 29/842,580, entitled “Staple Cartridge for Linear Surgical Stapler,” filed Jun. 14, 2022, the disclosure of which is incorporated by reference herein.

### C. Illustrative Use of Linear Surgical Stapler

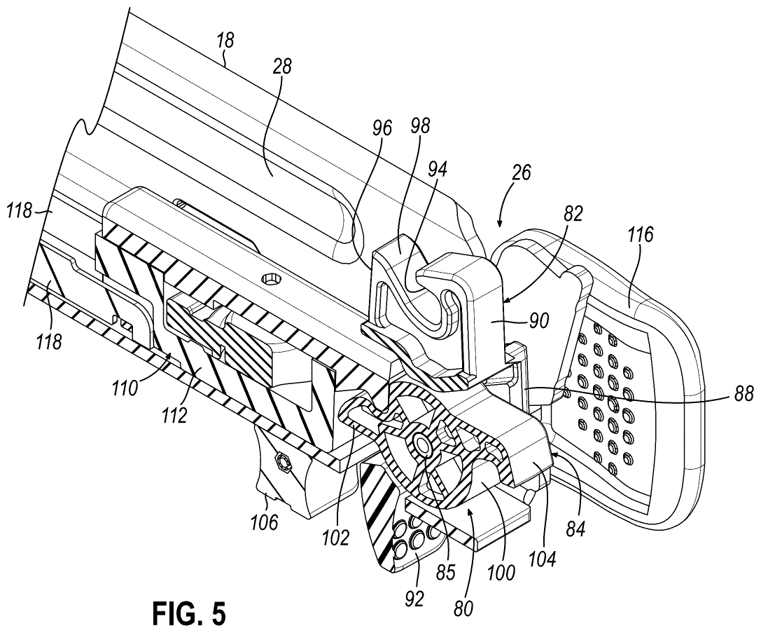

FIGS. 19A-19E show illustrative coupling of stapler halves (12, 14) and subsequent firing of assembled stapler (10) during a surgical procedure. As shown in FIG. 9A, clamp lever (40) of cartridge half (12) is provided in the open position so that jaw slots (50) align with vertical slots (24) of cartridge channel sidewalls (22). Additionally, firing assembly (110) is maintained in its proximal home position by detent (84) of retaining assembly (80), as shown in FIG. 5 described above. At this stage, a section of tissue (not shown) to be stapled and cut may be positioned over the top of staple cartridge (140) disposed in distal jaw portion (20) of cartridge half (12). Alternatively, the tissue may be positioned over staple cartridge (140) following coupling of the proximal ends of stapler halves (12, 14), described below.

As shown in FIGS. 19A-19B, the proximal ends of stapler halves (12, 14) are aligned with one another, and proximal anvil pin (70) is directed downwardly into proximal tapered notches (26) of cartridge channel (16) to engage latch finger (90) of anvil latch (82). This engagement forces anvil latch (82) to resiliently rotate clockwise, thus enabling latch finger (90) to capture anvil pin (70) and thereby releasably couple together the proximal ends of stapler halves (12, 14), as seen in FIG. 19B. With clamp lever (40) still in the open position as shown in FIG. 19B, stapler (10) is provided in a “hang-open” state such that stapler (10) may be held single-handedly by anvil half (14) while cartridge half (12) remains coupled to anvil half (14). As shown in FIG. 9C, and with clamp lever (40) remaining in the open position, anvil half (14) is rotated toward anvil half (14) about proximal anvil pin (70) so that distal clamp pin (68) of anvil half (14) is received into vertical slots (24) of cartridge channel sidewalls (22) and jaw slots (50) of clamp lever (40). Distal jaw portions (20, 64) of stapler halves (12, 14) are now in a partially approximated state such that tissue received therebetween may be finally adjusted before clamping.

As shown in FIG. 19D, clamp lever (40) is closed to draw anvil clamp pin (68) against the closed proximal ends of jaw slots (50) and thereby fully clamp anvil half (14) against cartridge half (12), with tissue (not shown) clamped between the stapling surfaces defined by staple cartridge (140) and anvil plate (72). A slight transverse gap is defined between staple cartridge (140) and anvil plate (72) by a tissue gap post (162) of staple cartridge (140), thus accommodating the tissue therebetween with a predetermined degree of tissue compression. As shown in FIGS. 19A and 19B, tissue gap post (162) is disposed at a distal end of staple cartridge (140) and is configured to contact a distal end of anvil plate (72) when stapler (10) is in the fully clamped state shown in FIG. 19D. In response to clamp lever (40) reaching the fully closed position, clamp lever latch (54) may rotate to capture a proximal end of a base wall of cartridge channel (16) and thereby assume a latched state in which clamp lever latch (54) maintains clamp lever (40) in the fully closed position.

As shown in FIG. 19E, upon reaching the fully clamped state, stapler (10) may be fired by driving a deployed actuator (114, 116) of firing assembly (110) distally along proximal frame portion (18) of cartridge half (12). This action causes elongate beams (118, 122) of firing assembly (110) to translate distally through corresponding channels formed in staple cartridge (140) and thereby fire staples into the clamped tissue via cam ramps (120) and staple drivers (172), and simultaneously cut the clamped tissue with knife (126). Following completion of the firing stroke, firing assembly (110) is returned to its proximal home position via the actuator (114, 116). Clamp lever latch (54) may then be depressed to release the proximal end of clamp lever (40) from cartridge channel (16), thus permitting clamp lever (40) to be re-opened. Then, release button (92) of retaining assembly (80) may be depressed to release anvil half (14) from cartridge half (12) so that stapler halves (12, 14) may be separated from one another, thereby releasing the newly stapled and severed tissue. It will be understood that in some versions, stapler (10) may include additional features to promote decoupling of stapler halves (12, 14), for example as disclosed in U.S. Pat. No. 11,033,266, incorporated by reference above.

### D. Linear Surgical Stapler Having Hang-Open Feature and Clamp Lever Latch Inhibiting Feature

As described above in connection with linear surgical stapler (10), a pivotable coupling is established between the proximal ends of stapler halves (12, 14) when anvil pin (70) is captured by anvil latch (82). Additionally, clamp lever latch (54) is configured to releasably capture a proximal end of a base wall of cartridge channel (16) to retain clamp lever (40) in its closed position during firing.

In some instances, it may be desirable to provide a linear surgical stapler with the ability to assume an open state in which the respective elongate bodies of the stapler halves assume a predetermined maximum angular orientation relative to one another and remain releasably coupled together at their proximal ends (referred to as a “hang-open” or “open aperture” state), such that the stapler in the assembled yet open state can be easily manipulated by an operator with a single hand. It may also be desirable to inhibit latching of the clamp lever latch when the firing assembly is displaced from its proximal home position, to serve as an indicator to a user that the staple cartridge may have already been at least partially fired and thus not suitable for use in a surgical procedure. The following description provides an illustrative example of a variation of linear surgical stapler (210) that provides such functionality.

FIG. 20 shows a proximal portion of another illustrative linear surgical stapler (210) that includes a cartridge half (212) and an anvil half (214), which are similar to cartridge half (12) and anvil half (14) of linear surgical stapler (10) described above except as otherwise described below. In particular, cartridge half (212) includes a clamp lever (240) having a clamp lever latch (254) that is configured to inhibit latching of the proximal end of clamp lever (240) to the proximal end of cartridge channel (216) when clamp lever (240) is closed unless firing assembly (310) is in its proximal home position. As shown in FIG. 21, clamp lever latch (254) includes a latch body (256) that pivotably couples with clamp lever (240), and a tapered protrusion (258) that extends upwardly from latch body (256). Tapered protrusion (258) includes a lower edge (260) configured to hook onto the proximal end of a base wall of cartridge channel (216) of cartridge half (212) and thereby retain clamp lever (240) in a closed state. Tapered protrusion (258) further includes a convexly curved cam surface (262) that faces distally.

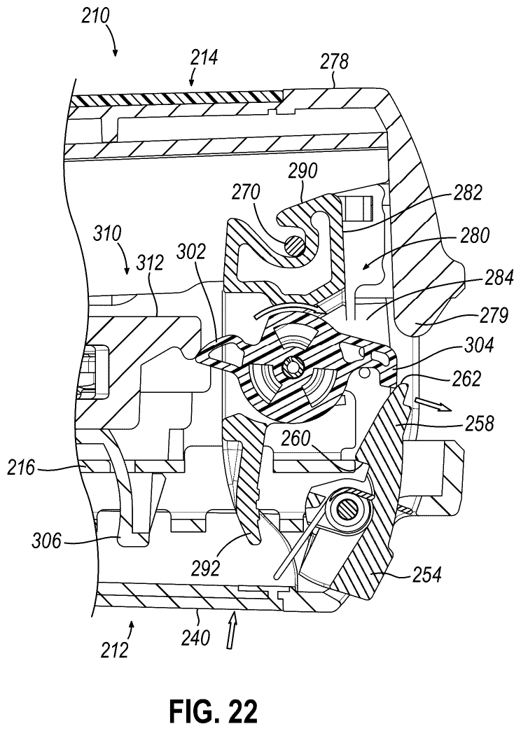

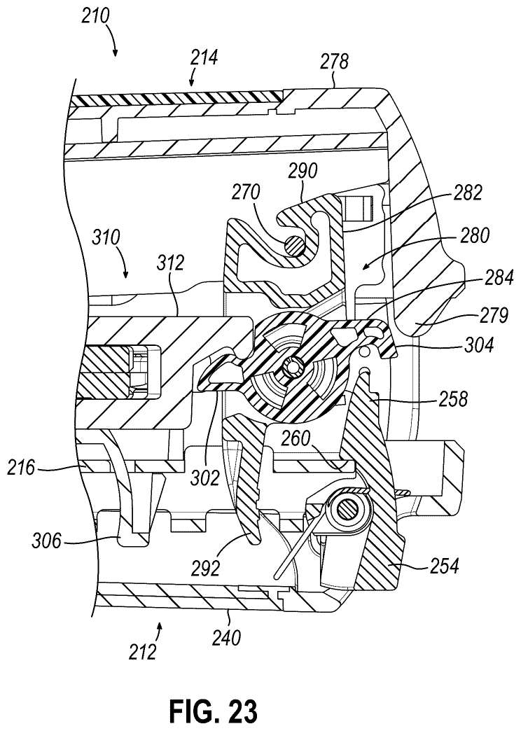

As shown in FIG. 22, when firing assembly (310) is displaced distally from its proximal home position, the proximal end of the firing assembly slide block (312) is disengaged from distal finger (302) of detent (284). Via the resilient bias of torsion spring (286), detent (284) rotates clockwise to a rotational position in which its proximal hook (304) is lowered. When clamp lever (240) is then moved from the open position to the closed position while firing assembly (310) is in this distally displaced state, the proximal end of proximal hook (304) contacts cam surface (262) of clamp lever latch (254). This camming interaction drives tapered protrusion (258) of clamp lever latch (254) away from cartridge channel (216) and thus prevents clamp lever latch (254) from latching onto the proximal end of cartridge channel (216). This result may alert the user that firing assembly (310) is displaced from its proximal home position such that staple cartridge (140) may already be partially fired and thus not suitable for use on a patient. In response, the user may then return the firing assembly (310) to its proximal home position, substitute in a fresh staple cartridge (140), and proceed with the surgical procedure. With firing assembly (310) in its proximal home position as shown in FIG. 23, detent (284) is maintained by slide block (312) in a counter-clockwise rotational position in which proximal hook (104) is raised. Accordingly, clamp lever latch (254) may avoid contact with proximal hook (304) and latch onto the proximal end of cartridge channel (216) when clamp lever (240) is closed.

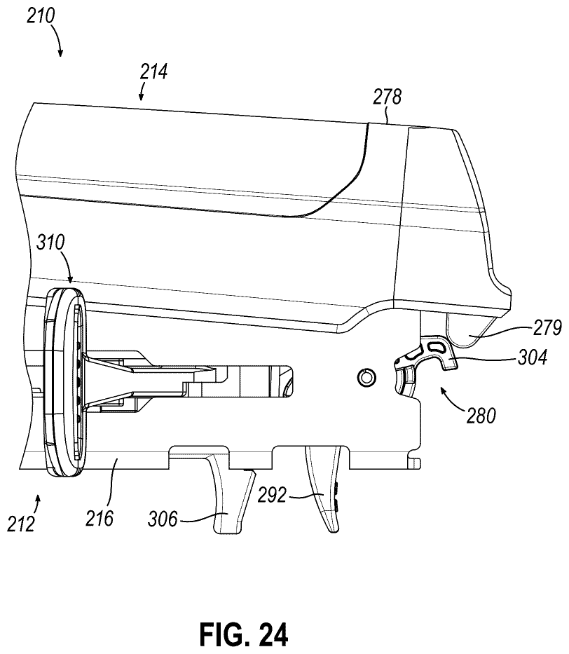

As shown in FIG. 24, anvil half (214) of stapler (210) further includes an anvil shroud (278) having a protrusion in the form of a rib (279) that extends downwardly from a proximal end of anvil shroud (278). Rib (279) includes a rounded tip that is configured to contact a proximal surface of proximal hook (304) of retaining assembly (280) of cartridge half (212) when stapler (210) is held by anvil half (214) in a hang-open state similar to the hang-open state shown in FIG. 19B. The engagement between rib (279) and proximal hook (304) of detent (284) in the hang-open state provides a resistance to decoupling of the stapler halves (212, 214). More specifically, in the hang-open state, proximal anvil pin (270) exerts an upward prying force against the underside latching surface (294) of latch finger (290) of anvil latch (282), which causes anvil latch (282) to rotate clockwise release anvil pin (270) and decouple stapler halves (212, 214). The interaction between rib (279) and proximal hook (304) of detent (284) effectively counteracts and lessens this upward prying force, thereby providing resistance to decoupling of stapler halves (212, 214) and enabling stapler (210) to remain in the hang-open state more securely without unintentional decoupling. Such a configuration may be particularly beneficial for versions of stapler (210) in which cartridge half (212) has an increased weight, due to size and/or material selection, that results in an increase prying force being exerted between proximal anvil pin (270) and anvil latch (282) in the hang-open state.

E. Linear Surgical Stapler Having Clamp Lever with Mis-Clamp Indicator

As described above in connection with linear surgical stapler (10), when clamp pin (68) is properly aligned with jaw slots (50), actuation of clamp lever (40) from the open position to the closed position (see FIGS. 19C-19D) operates to cammingly drive the opposed lateral ends of clamp pin (68) proximally within jaw slots (50) and thereby clamp stapler halves (12, 14) together to approximate stapling surfaces (72, 140). In some instances, it may be difficult for the surgeon to sufficiently approximate stapler halves (12, 14) to properly align clamp pin (68) with jaw slots (50), such as when clamping on thick tissue, and the surgeon may unknowingly close clamp lever (40) without properly capturing clamp pin (68), such that stapler halves (12, 14) are not properly clamped together (referred to as a “mis-clamped” state). Accordingly, it may be desirable to provide stapler (10) with one or more features that are configured provide to the surgeon a clear indication when clamp pin (68) has not been properly captured within jaw slots (50), and that readjustment of clamp lever (40) is required to achieve proper clamping on tissue.

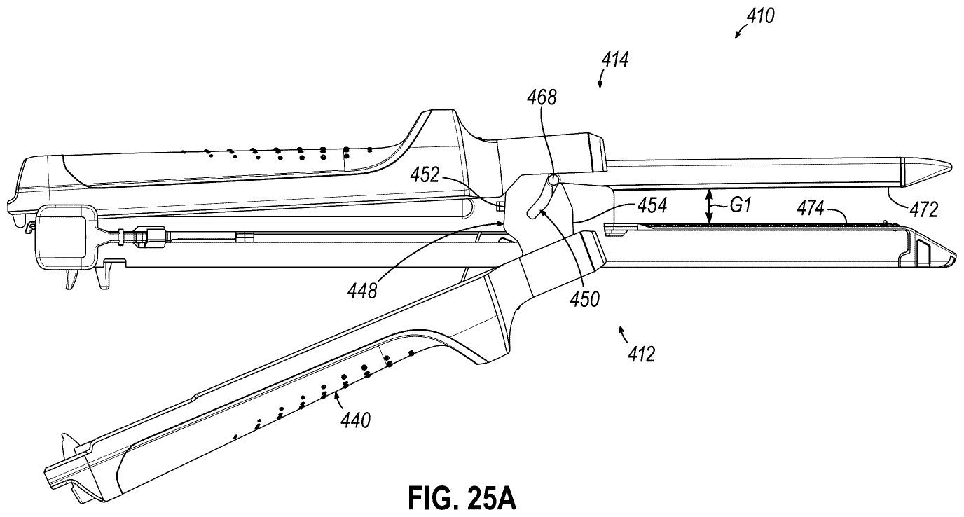

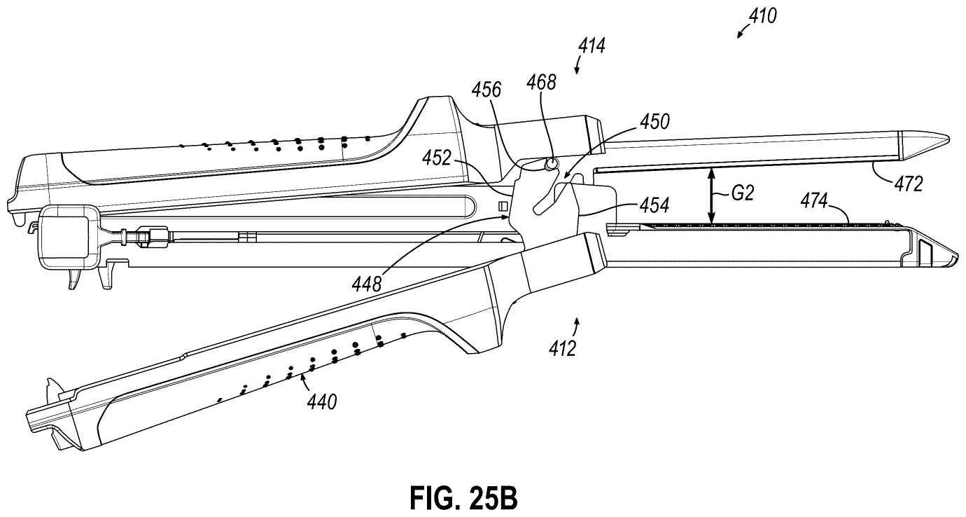

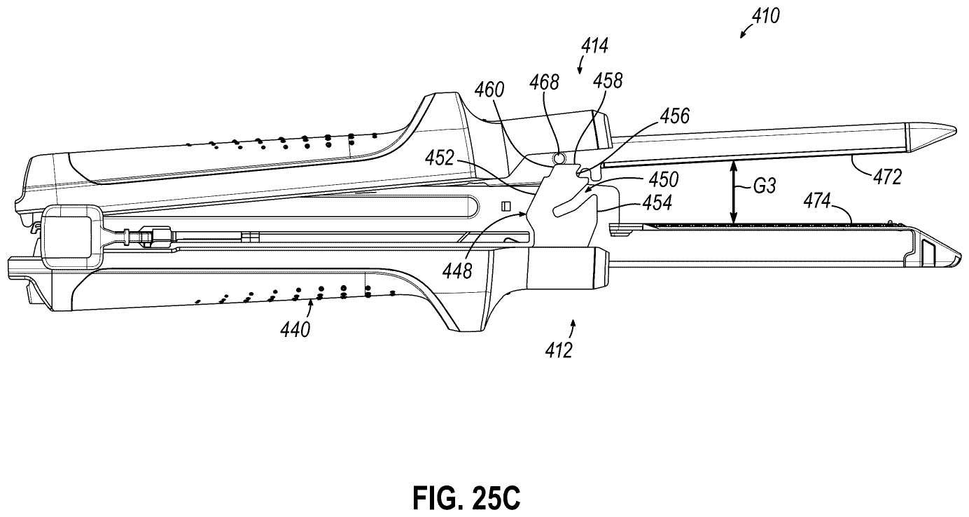

FIGS. 25A-25C show another illustrative linear surgical stapler (410) that includes a cartridge half (412) and an anvil half (414) that are similar to cartridge half (12) and anvil half (14) of linear surgical stapler (10) described above except as otherwise described below. In particular, clamp lever (440) of cartridge half (412) includes a laterally opposed pair of jaws (448) that are uniquely shaped to provide to a surgeon a clear indication when clamp pin (68) has not been properly aligned with and captured by jaw slots (450) during closure of clamp lever (440).

Referring briefly to FIG. 25C, each jaw (448) includes a first pin-receiving opening in the form of an arcuate slot (450) having a closed proximal end and an open distal end configured to receive a respective lateral end of clamp pin (68). Each jaw slot (450) divides its respective jaw (448) into an upper jaw portion (452) and a lower jaw portion (454) that are interconnected along a proximal side of jaw (448). Each upper jaw portion (452) at its distal end includes a second pin-receiving opening in the form of a recess (456) (also referred to as a hook) that is generally semi-circular in shape and is sized to releasably capture a respective lateral end of clamp pin (68) when clamp pin (68) is mis-aligned with jaw slots (450), as described further below. As shown, each recess (456) is offset and spaced apart from the respective arcuate slot (450) such that slot (450) and recess (456) do not communicate with one another. Additionally, upper jaw portion (452) has an angled outer profile such that a width of upper jaw portion (452) measured transversely between jaw slot (450) and an outer surface (458) increases proximally from a distal end of jaw (448) toward an apex (460) of upper jaw portion (452) that is approximately aligned with a mid-length point of slot (450), before then decreasing. Accordingly, a maximum such width of upper jaw portion (452) is defined at apex (460). As described in greater detail below, each of recess (456) and outer surface (458) is configured to engage clamp pin (68) when clamp pin (68) is misaligned with jaw slots (450) so as to indicate to the surgeon that clamp pin (68) has not been properly captured by jaw slots (450) and that readjustment of stapler halves (412, 414) is required.

FIG. 25A shows an illustrative scenario in which stapler halves (412, 414) have been sufficiently approximated such that clamp pin (68) is properly aligned with and capturable by jaw slots (450). Stapling surfaces (472, 474) are situated in a partially approximated state that defines a gap (G1) at a mid-length point of the staple cartridge. In this scenario, clamp lever (440) may then be closed to properly clamp stapler halves (412, 414) together and fully approximate stapling surfaces (472, 474), such as shown in FIGS. 19C-19D in connection with linear surgical stapler (10).

FIG. 25B shows an alternative scenario in which a surgeon has failed sufficiently approximate stapler halves (412, 414) during clamping such that clamp pin (68) is just slightly misaligned with and not capturable by jaw slots (450). When the surgeon then attempts to close clamp lever (440), clamp pin (68) is captured by jaw recesses (456) such that clamp lever (440) is inhibited from closing and is constrained in an intermediate position between its open position and its closed position. This situates stapling surfaces (472, 474) in a spaced-apart state in which they define a second gap (G2) at the mid-length point of the staple cartridge, where the second gap (G2) is noticeably larger than the first gap (G1) of FIG. 25A. This provides the surgeon with a visible and tactile indication that clamp lever (440) is not properly aligned with clamp pin (68) and must be reopened and adjusted to achieve proper clamping together of stapler halves (412, 414).

FIG. 25C shows yet another alternative scenario in which a surgeon has failed to sufficiently approximate stapler halves (412, 414) during clamping such that clamp pin (68) is more substantially misaligned with and not capturable by jaw slots (450). In this scenario, clamp lever (440) has been permitted to pivot to its closed position while clamp pin (68) has simultaneously tracked proximally along the outer surface (458) of each upper jaw portion (452), via rotation of jaw (448) relative to clamp pin (468), and ultimately rest at apex (460) when clamp lever (440) is fully closed. The angled outer profile of upper jaw portions (452) acts as a camming feature that urges the distal end of anvil half (412) away from the distal end of cartridge half (412) as clamp lever (440) is closed clamp pin (68) approaches apex (460) along outer surface (458). This situates stapling surfaces (472, 474) in an even further spaced-apart state in which they define a third gap (G3) at the mid-length point of the staple cartridge, where the third gap (G3) is noticeably larger than the second gap (G2) of FIG. 25B. Thus, the surgeon is provided with a visible indication that stapler (410) is not properly clamped and that clamp lever (440) must be reopened to properly approximate stapler halves (412, 414) so that clamp pin (68) may be properly aligned with and received by jaw slots (450) before clamp lever (440) is reclosed.

### F. Linear Surgical Stapler Having Pin Blocking Protrusion

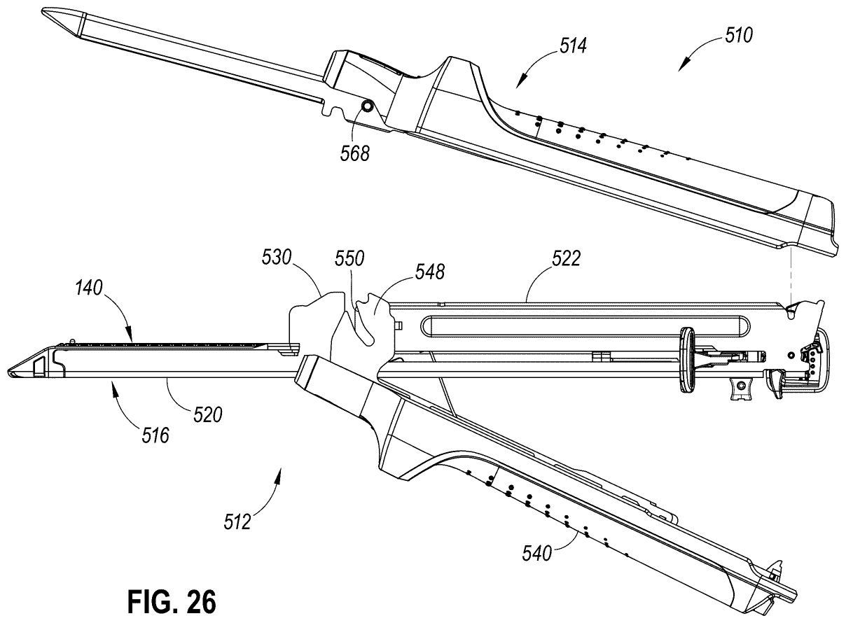

FIGS. 26-28 show another illustrative linear surgical stapler (510) that includes a cartridge half (512) and an anvil half (514) and is similar in structure and function to linear surgical staplers (10, 210, 410) described above except as otherwise described below. Notably, cartridge half (512) includes a unique cartridge channel (516) that is configured to block distal translation of clamp pin (568) relative to cartridge half (512), and thus full closure of clamp lever (540), when stapler (510) is in a mis-clamped state in which clamp pin (568) is captured by jaw recesses (556) instead of jaw slots (550). As described below, such a configuration ensures that clamp pin (568) and adjacent components of anvil half (514) coupled with clamp pin (568) remain intact and non-compromised if and when a user erroneously attempts to exert additional closure force on clamp lever (540) in the mis-clamped state.

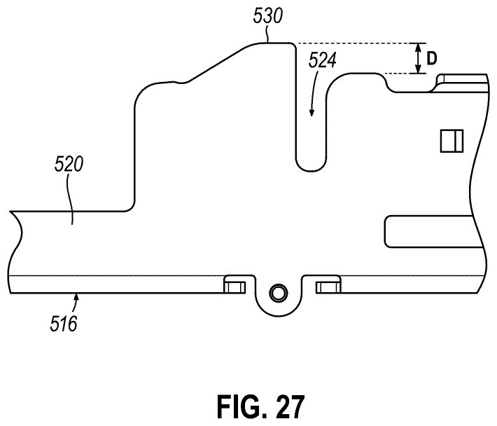

As shown best in FIG. 27, a medial portion of cartridge channel (516) includes a pair of blocking protrusions (530) located longitudinally between the distal sides of sidewall slots (524) and a proximal end of distal jaw portion (520). Blocking protrusions (530) extending upwardly and are raised relative to the adjacent portions of channel sidewalls (522) such that a top-most surface of each blocking protrusion (530) distal to sidewall slot (524) is elevated above a top-most surface of the respective portion of sidewall (522) immediately proximal to the respective sidewall slot (524) by a height difference (D) that is approximately equal to an outer diameter of clamp pin (568). Blocking protrusions (530) of the present version are formed as integral features of cartridge channel (516), though in other versions blocking protrusions (530) may be manufactured separately from and then affixed to cartridge channel (516).

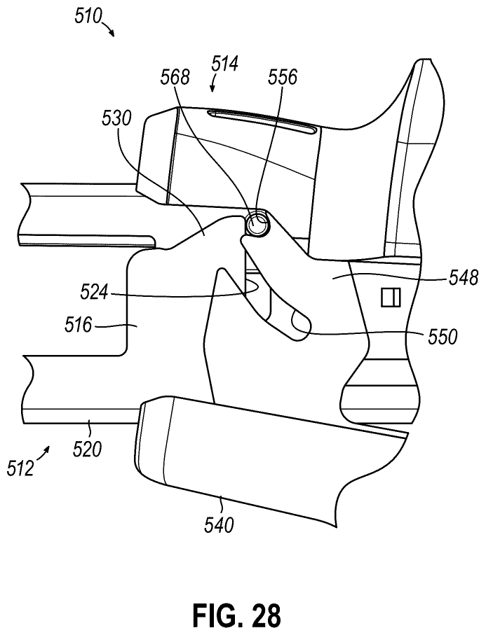

FIG. 28 shows clamp lever (540) of linear surgical stapler (510) in a partially-closed, mis-clamped state similar to the state of linear surgical stapler (410) shown in FIG. 25B. In particular, stapler (510) is shown after a user has partially approximated stapler halves (512, 514) and then partially closed clamp lever (540) without first ensuring that clamp pin (568) is aligned with arcuate slots (550) of clamp lever jaws (548). As a result, clamp pin (568) is misaligned with clamp lever jaw slots (550) such that its lateral ends are instead positioned within clamp lever jaw recesses (556). As similarly described above in connection with stapler (410) of FIGS. 25A-25C, this engagement of clamp pin (568) with clamp lever jaw recesses (556) provides a visible and tactile indication to the user that clamp lever (540) is not properly aligned with clamp pin (568) and must be reopened and adjusted to achieve proper clamping of stapler halves (512, 514).

As shown in FIG. 28, clamp pin (568) is constrained in a proximal direction by a distally facing surface (defined by recesses (556)) of each clamp lever jaw (548), and in a distal direction by a proximally facing surface of each blocking protrusion (530), where a central axis of clamp pin (568) is positioned beneath a top-most surface of each blocking protrusion (530). Accordingly, even if the user erroneously continues to apply a closure force to clamp lever (540) in this mis-clamped state, clamp pin (568) (and anvil half (514) as a whole) is blocked from translating distally relative to cartridge channel (516), and clamp lever (540) is therefore inhibited from fully closing. Such constraint of clamp pin (568) insulates internal components of stapler (510) from the closure force erroneously applied by the user to clamp lever (540) in the mis-clamped state, and therefore ensures the integrity of such internal components.

Additionally, in the scenario that the user failed to couple together the proximal ends of stapler halves (512, 514) before placing clamp lever (540) in the mis-clamped state, the longitudinal constraint of clamp pin (568) by blocking protrusions (530) may encourage the proximal ends to couple together in response to the additional closure force applied by the user to clamp lever (540). Accordingly, the user may then reopen clamp lever (540) and properly align it with clamp pin (568) to achieve a proper clamping of stapler halves (512, 514).

Any of the illustrative linear surgical staplers (10, 210, 410, 510) disclosed herein may be further configured in accordance with any one or more teachings of U.S. Pat. Pub. No. 2023/0397911, entitled “Linear Surgical Stapler,” published Dec. 14, 2023; and/or U.S. Pat. Pub. No. 2024/0058002, entitled “Linear Surgical Stapler,” published Feb. 22, 2024, the disclosures of which are incorporated by reference herein in their entireties.

### II. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical stapler, comprising: (a) a first elongate body having a distal portion configured to present a first stapling surface; (b) a second elongate body having a distal portion configured to present a second stapling surface, wherein the first and second elongate bodies are configured to releasably couple together to enable the first and second stapling surfaces to cooperate to clamp and staple tissue with a plurality of staples; (c) a clamp protrusion extending from the first elongate body; (d) a clamp lever movably coupled with the second elongate body, wherein the clamp lever includes an opening configured to releasably capture and draw the clamp protrusion toward the second elongate body as the clamp lever advances from an open position toward a closed position to thereby approximate the first and second stapling surfaces; and (e) a blocking protrusion positioned to block the clamp protrusion from translating distally relative to the first elongate body when the clamp protrusion is misaligned with the opening of the clamp lever.

### Example 2

The surgical stapler of Example 1, wherein the blocking protrusion is defined by the second elongate body.

### Example 3

The surgical stapler of any of the preceding Examples, wherein the second elongate body includes: (i) an opening configured to receive the clamp protrusion as the first and second elongate bodies approximate, (ii) a first sidewall portion extending proximally from a proximal side of the opening, and (iii) a second sidewall portion extending distally from distal side of the opening, wherein the second sidewall portion defines the blocking protrusion.

### Example 4

The surgical stapler of Example 3, wherein the distal sidewall portion is taller than the proximal sidewall portion.

### Example 5

The surgical stapler of Example 4, wherein a tallest surface of the distal sidewall portion is raised relative to a tallest surface of the proximal sidewall portion.

### Example 6

The surgical stapler of any of the preceding Examples, wherein the opening of the clamp lever comprises a first clamp lever opening, wherein the clamp lever further includes a second clamp lever opening offset from the first clamp lever opening in non-communication relation, wherein the second clamp lever opening is configured to releasably capture the clamp protrusion when the clamp protrusion is misaligned with the first opening.

### Example 7

The surgical stapler of Example 6, wherein the clamp lever is inhibited from assuming the closed position when the clamp protrusion is positioned within the second clamp lever opening.

### Example 8

The surgical stapler of any of Examples 6 through 7, wherein the first clamp lever opening is configured to receive the clamp protrusion when the clamp lever is oriented at a first angle relative to a proximal body portion of the second elongate body, wherein the second clamp lever opening is configured to receive the clamp protrusion when the clamp lever is oriented at a second angle relative to the proximal body portion, wherein the second angle is less than the first angle.

### Example 9

The surgical stapler of Example 8, wherein the first clamp lever opening comprises an arcuate slot, wherein the second clamp lever opening comprises a recess.

### Example 10

The surgical stapler of any of Examples 6 through 9, wherein the clamp lever and the blocking protrusion are configured to cooperate to constrain the clamp protrusion in proximal and distal directions and thereby inhibit proximal and distal movement of the clamp protrusion relative to the second elongate body when the clamp protrusion is positioned within the second lever opening.

### Example 11

The surgical stapler of any of the preceding Examples, wherein proximal ends of the first and second elongate bodies are configured to releasably couple together, wherein the blocking protrusion is configured to inhibit closure of the clamp lever when the proximal ends are decoupled from one another.

### Example 12

The surgical stapler of Example 11, wherein the blocking protrusion is configured to facilitate coupling of the proximal ends with one another in response to a closure force applied to the clamp lever by a user while the proximal ends are decoupled from one another.

### Example 13

The surgical stapler of any of Examples 11 through 12, wherein the proximal end of the first elongate body includes a fixed latch protrusion, and the proximal end of the second elongate body includes a resiliently biased latch configured to releasably capture the fixed latch protrusion and thereby couple the proximal ends of the first and second elongate bodies.

### Example 14

The surgical stapler of any of the preceding Examples, wherein the clamp protrusion comprises a pin.

### Example 15

The surgical stapler of any of the preceding Examples, wherein the distal portion of the first elongate body supports an anvil that defines the first stapling surface and has a plurality of staple forming pockets, wherein the distal portion of the second elongate body is configured to support a stapling assembly that defines the second stapling surface and has a plurality of staples configured to be ejected toward and formed by the staple forming pockets.

### Example 16

A surgical stapler, comprising: (a) a first elongate body having a distal portion configured to present a first stapling surface; (b) a second elongate body having a distal portion configured to present a second stapling surface, wherein the first and second elongate bodies are configured to releasably couple together to enable the first and second stapling surfaces to cooperate to clamp and staple tissue with a plurality of staples; (c) a clamp protrusion extending from the first elongate body; (d) a clamp lever movably coupled with the second elongate body, wherein the clamp lever includes: (i) a first opening configured to releasably capture and draw the clamp protrusion toward the second elongate body as the clamp lever advances from an open position toward a closed position to thereby approximate the first and second stapling surfaces, and (ii) a second opening offset from the first opening and configured to releasably capture the clamp protrusion when the clamp protrusion is misaligned with the first opening; and (e) a blocking protrusion extending from the second elongate body and configured to block the clamp protrusion from translating distally relative to the first elongate body when the clamp protrusion is disposed within the second opening.

### Example 17

The surgical stapler of Example 16, wherein the blocking protrusion is defined by the second elongate body.

### Example 18

The surgical stapler of any of Examples 16 through 17, wherein the second opening is proximal to the first opening.

### Example 19

A surgical stapler, comprising: (a) a first elongate body having a distal portion configured to present a first stapling surface; (b) a second elongate body having a distal portion configured to present a second stapling surface and configured to releasably couple with the first elongate body to enable the first and second stapling surfaces to cooperate to clamp and staple tissue with a plurality of staples, wherein the second elongate body includes: (i) an opening, (ii) a first sidewall portion extending proximally from a proximal side of the opening, and (iii) a second sidewall portion extending distally from a distal side of the opening, wherein the distal sidewall portion is taller than the proximal sidewall portion, (c) a clamp protrusion extending from the first elongate body and configured to be received within the opening of the second elongate body as the first and second elongate bodies approximate; and (d) a clamp lever movably coupled with the second elongate body, wherein the clamp lever includes: (i) a first clamp lever opening configured to releasably capture and draw the clamp protrusion toward the second elongate body as the clamp lever advances from an open position toward a closed position to thereby approximate the first and second stapling surfaces, and (ii) a second clamp lever opening offset from the first clamp lever opening and configured to releasably capture the clamp protrusion when the clamp protrusion is misaligned with the first clamp lever opening, wherein the distal sidewall portion of the second elongate body is configured to inhibit distal translation of the first elongate body relative to the second elongate body when the clamp protrusion is misaligned with the first clamp lever opening.

### Example 20

The surgical stapler of Example 19, wherein a top-most surface of the distal sidewall portion is raised relative to a top-most surface of the proximal sidewall portion.

### III. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Further, any one or more teachings disclosed herein may be combined with any one or more teachings disclosed in U.S. Pat. No. 10,631,866, entitled “Release Mechanism for Linear Surgical Stapler,” issued Apr. 28, 2020; U.S. Pat. No. 10,667,818, entitled “Lockout Assembly for Linear Surgical Stapler,” issued Jun. 2, 2020; U.S. Pat. No. 10,932,781, entitled “Features to Align and Close Linear Surgical Stapler,” issued Mar. 2, 2021; U.S. Pat. No. 10,898,197, entitled “Releasable Coupling Features for Proximal Portions of Linear Surgical Stapler,” issued Jan. 26, 2021; U.S. Pat. No. 10,874,398, entitled “Firing Lever Assembly for Linear Surgical Stapler,” issued Dec. 29, 2020; U.S. Pat. No. 10,687,819, entitled “Clamping Mechanism for Linear Surgical Stapler,” issued Jun. 23, 2020; U.S. Pat. No. 10,898,187, entitled “Firing System for Linear Surgical Stapler,” issued Jan. 26, 2021; U.S. Pat. No. 11,033,266, entitled “Decoupling Mechanism for Linear Surgical Stapler, issued Jun. 15, 2021; U.S. Pat. No. 11,045,193, entitled “Anvil Assembly for Linear Surgical Stapler,” issued Jun. 29, 2021; U.S. Pat. No. 10,905,419, entitled “Closure Assembly for Linear Surgical Stapler,” issued Feb. 2, 2021; U.S. Pat. No. 11,278,285, entitled “Clamping Assembly for Linear Surgical Stapler,” issued Mar. 22, 2022; U.S. Pat. No. 11,229,433, entitled “Linear Surgical Stapler,” issued Jan. 25, 2022; U.S. Pat. No. 12,016,555, entitled “Method of Forming an Anvil for a Surgical Stapler,” issued Jun. 25, 2024; U.S. Pat. No. 11,224,425, entitled “Surgical Linear Cutter Wishbone Separation Mechanism with Detent,” issued Jan. 18, 2022; U.S. Pat. No. 11,219,454, entitled “Pin Trap Mechanism for Surgical Linear Cutter,” issued Jan. 11, 2022; U.S. Pat. No. 11,399,827, entitled “Separation Mechanism for Surgical Linear Cutter,” issued Aug. 2, 2022; U.S. Pat. No. 11,937,812, entitled “Lockout Feature for Linear Surgical Stapler Cartridge,” issued Mar. 26, 2024; U.S. patent application Ser. No. 29/842,580, entitled “Staple Cartridge for Linear Surgical Stapler,” filed Jun. 14, 2022; and/or U.S. patent application Ser. No. 29/842,581, entitled “Linear Surgical Stapler,” filed Jun. 14, 2022. The disclosure of each of these references is incorporated by reference herein, in its entirety.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A surgical stapler, comprising:
(a) a first elongate body having a distal portion configured to present a first stapling surface;
(b) a second elongate body having a distal portion configured to present a second stapling surface, wherein the first and second elongate bodies are configured to releasably couple together to enable the first and second stapling surfaces to cooperate to clamp and staple tissue with a plurality of staples, wherein the second elongate body defines a longitudinal axis, wherein the second elongate body includes a distal sidewall and a proximal sidewall, wherein the distal sidewall and the proximal sidewall defines an opening, wherein the distal sidewall is longer than the proximal sidewall in a direction perpendicular to the longitudinal axis;
(c) a clamp protrusion extending from the first elongate body;
(d) a clamp lever movably coupled with the second elongate body, wherein the clamp lever includes an opening configured to releasably capture and draw the clamp protrusion toward the second elongate body as the clamp lever advances from an open position toward a closed position to thereby approximate the first and second stapling surfaces; and
(e) a blocking protrusion positioned to block the clamp protrusion from translating distally relative to the first elongate body when the clamp protrusion is misaligned with the opening of the clamp lever, wherein a portion of the distal sidewall is defined by the blocking protrusion.

2. The surgical stapler of claim 1, wherein the blocking protrusion is defined by the second elongate body.

3. The surgical stapler of claim 2, wherein:
(i) the opening is configured to receive the clamp protrusion as the first and second elongate bodies approximate,
(ii) the proximal sidewall includes a first sidewall portion extending proximally from a proximal side of the opening, and
(iii) the distal sidewall includes a second sidewall portion extending distally from a distal side of the opening, wherein the second sidewall portion defines the blocking protrusion.

4. The surgical stapler of claim 3, wherein the second sidewall portion is taller than the first sidewall portion.

5. The surgical stapler of claim 4, wherein a tallest surface of the second sidewall portion is raised relative to a tallest surface of the first sidewall portion.

6. The surgical stapler of claim 1, wherein the opening of the clamp lever comprises a first clamp lever opening, wherein the clamp lever further includes a second clamp lever opening offset from the first clamp lever opening in non-communication relation, wherein the second clamp lever opening is configured to releasably capture the clamp protrusion when the clamp protrusion is misaligned with the first opening.

7. The surgical stapler of claim 6, wherein the clamp lever is inhibited from assuming the closed position when the clamp protrusion is positioned within the second clamp lever opening.

8. The surgical stapler of claim 6, wherein the first clamp lever opening is configured to receive the clamp protrusion when the clamp lever is oriented at a first angle relative to a proximal body portion of the second elongate body, wherein the second clamp lever opening is configured to receive the clamp protrusion when the clamp lever is oriented at a second angle relative to the proximal body portion, wherein the second angle is less than the first angle.

9. The surgical stapler of claim 8, wherein the first clamp lever opening comprises an arcuate slot, wherein the second clamp lever opening comprises a recess.

10. The surgical stapler of claim 6, wherein the clamp lever and the blocking protrusion are configured to cooperate to constrain the clamp protrusion in proximal and distal directions and thereby inhibit proximal and distal movement of the clamp protrusion relative to the second elongate body when the clamp protrusion is positioned within the second lever opening.

11. The surgical stapler of claim 1, wherein proximal ends of the first and second elongate bodies are configured to releasably couple together, wherein the blocking protrusion is configured to inhibit closure of the clamp lever when the proximal ends are decoupled from one another.

12. The surgical stapler of claim 11, wherein the blocking protrusion is configured to facilitate coupling of the proximal ends with one another in response to a closure force applied to the clamp lever by a user while the proximal ends are decoupled from one another.

13. The surgical stapler of claim 11, wherein the proximal end of the first elongate body includes a fixed latch protrusion, and the proximal end of the second elongate body includes a movable latch configured to releasably capture the fixed latch protrusion and thereby couple the proximal ends of the first and second elongate bodies.

14. The surgical stapler of claim 1, wherein the clamp protrusion comprises a pin.

15. The surgical stapler of claim 1, wherein the distal portion of the first elongate body supports an anvil that defines the first stapling surface and has a plurality of staple forming pockets, wherein the distal portion of the second elongate body is configured to support a stapling assembly that defines the second stapling surface and has a plurality of staples configured to be ejected toward and formed by the staple forming pockets.

16. A surgical stapler, comprising:
(a) a first elongate body having a distal portion configured to present a first stapling surface;
(b) a second elongate body having a distal portion configured to present a second stapling surface, wherein the first and second elongate bodies are configured to releasably couple together to enable the first and second stapling surfaces to cooperate to clamp and staple tissue with a plurality of staples;
(c) a clamp protrusion extending from the first elongate body;
(d) a clamp lever movably coupled with the second elongate body about a pivot axis, wherein the clamp lever includes:
(i) a first opening movable about the pivot axis to thereby be configured to releasably capture and draw the clamp protrusion toward the second elongate body as the clamp lever advances from an open position toward a closed position to thereby approximate the first and second stapling surfaces, and
(ii) a second opening offset from the first opening and movable about the pivot axis to thereby be configured to releasably capture the clamp protrusion when the clamp protrusion is misaligned with the first opening; and
(e) a blocking protrusion extending from the second elongate body and configured to block the clamp protrusion from translating distally relative to the first elongate body when the clamp protrusion is disposed within the second opening.

17. The surgical stapler of claim 16, wherein the blocking protrusion is defined by the second elongate body.

18. The surgical stapler of claim 16, wherein the second opening is proximal to the first opening.

19. A surgical stapler, comprising:
(a) a first elongate body having a distal portion configured to present a first stapling surface;
(b) a second elongate body having a distal portion configured to present a second stapling surface and configured to releasably couple with the first elongate body to enable the first and second stapling surfaces to cooperate to clamp and staple tissue with a plurality of staples, wherein the second elongate body includes:
(i) an opening,
(ii) a first sidewall portion extending proximally from a proximal side of the opening, and
(iii) a second sidewall portion extending distally from a distal side of the opening, wherein the second sidewall portion is taller than the first sidewall portion in a direction that is perpendicular to a proximal-to-distal direction of the second elongate body,
(c) a clamp protrusion extending from the first elongate body and configured to be received within the opening of the second elongate body as the first and second elongate bodies approximate; and
(d) a clamp lever movably coupled with the second elongate body, wherein the clamp lever includes:
(i) a first clamp lever opening configured to releasably capture and draw the clamp protrusion toward the second elongate body as the clamp lever advances from an open position toward a closed position to thereby approximate the first and second stapling surfaces, and
(ii) a second clamp lever opening offset from the first clamp lever opening and configured to releasably capture the clamp protrusion when the clamp protrusion is misaligned with the first clamp lever opening,
wherein the distal sidewall portion of the second elongate body is configured to inhibit distal translation of the first elongate body relative to the second elongate body when the clamp protrusion is misaligned with the first clamp lever opening.

20. The surgical stapler of claim 19, wherein a top-most surface of the distal sidewall portion is raised relative to a top-most surface of the first sidewall portion.
